(19) United States
(12) Patent Application Publication
(10) Pub. No.: US 2013/0319880 A1
(43) Pub. Date: Dec. 5, 2013

(54) Impedimetric Biosensor System With Improved Sensing Efficiency

(71) Applicants: Ching-Chou Wu, Taichung City (TW); Dong-Jie Yang, Pingtung County (TW)

(72) Inventors: Ching-Chou Wu, Taichung City (TW); Dong-Jie Yang, Pingtung County (TW)

(21) Appl. No.: 13/487,467

(22) Filed: Jun. 4, 2012

(51) Int. Cl. G01N 33/50

(52) U.S. Cl. 205/792; 204/403.01

(57) ABSTRACT

Provided is an impedimetric biosensor system having a chip and an electrochemical sensor connected to the chip. The chip includes a substrate and at least one electrode assembly. The electrode assembly is mounted on the substrate as working electrodes for contacting an analyte. The electrode assembly is controlled under a controlling condition for alternating current electroosmotic flow (ACEOF), such that an ACEO vortex occurs to increase collision between a target in the analyte and the at least one electrode assembly. The impedimetric biosensor system has an improved efficiency on detecting a target analyte.

### BACKGROUND OF THE INVENTION

1. Field of the Invention

The present invention relates to a biosensor system, particularly to an impedimetric biosensor system with improved sensing efficiency by alternating current-electroosmotic flow (ACEOF).

2. Description of the Prior Arts

A common technique for detecting biomolecules is affinity biosensor, which measures the variance in configuration, electric charge, impedance, mass, heat energy or spatial hindrance before and after the occurrence of affinity binding between receptor and ligand, and antibody and antigen, or hybridization between two nucleic acids. Compared with detection of biomolecules by high performance liquid chromatography (HPLC) and enzyme-linked immunosorbent assay (ELISA) or other methods, detection with biosensor by affinity binding is more economic and laborsaving. However, reaction efficiency of the affinity binding between probe and analyte in the current techniques depends on diffusion driven by concentration gradient. Accordingly, the reaction between probes and analytes usually requires more than one hour to reach the reaction plateau. Since the analyte is usually extremely tiny or in a rare amount, the detection limit of existing biosensor cannot be further lowered.

Label-free detecting approaches, such as micromechanical cantilever-based technique, quartz crystal microbalance, surface plasmon resonance spectroscopy and electrochemical impedance spectroscopy (EIS) allow detection for biomolecules to be simpler, faster and more cost-effective, wherein the EIS can be used to determine the concentration of the target biomolecule of the analyte by measuring the changes in the electron transfer resistance on the surface and the capacitance of the electric double layer (EDL). Although label-free detecting approaches by impedimentary method can significantly save the time for detection, hour-long reaction time between probe and analyte still depends on diffusion in the stationary solution and limits the detection efficiency.

In order to improve the detection efficiency, the biosensor based on affinity binding was combined with convective transport parts through ACEOF, dielectrophoresis (DEP), electrothermal effect (ELE) and induced-charge electroosmosis (ICEO) to modulate the movement of liquid, gel beads or other miniature substances. However, the requirement of bulky pumping equipment and connecting channels increases the cost and prepared procedures of detection. In contrast, the use of AC electrokinetics including DEP, ETE and electroosmosis (EO) is beneficial for the manipulation of particles and fluid in a micro analysis system, which does not need external pumps and valves.

However, DEP force is not suitable for directly manipulating the tens of base single strain DNA of nanometer-scale size due to the DEP force proportional to the particle volume. Moreover, the production of an obvious ETE flow (about 100 μm/s) needs high conductivity electrolyte (typically >1 mS/cm), high frequency (>100 kHz) and large driving voltage (about root-mean-square voltage of 7 Volts). The high voltage may induce Faradaic reaction to destroy the thin-film electrodes and the surface modification layer of the biosensor. With regards to ACEO, to the best of our knowledge the impedimetric biosensor integrated with ACEO stirring for the biomolecule detection has not been investigated.

The principle of the ACEO is known in the art. As shown in FIG. 9, when an alternating current was applied between the two electrodes, electrolytes between the electrodes are affected by the static electricity to form EDL on the surface of the electrode. The oppositely electric charges, also called counterions, are respectively accumulated on the surfaces of the electrodes, which is caused by gradient of the alternating electric field. Accordingly, Coulomb force in a direction outward the central of the electrodes is formed and induces a vortex driven by the movement of hydrated counterions in EDL. The phenomenon is the so-called ACEOF.

To overcome the shortcomings, the present invention provides an impedimetric biosensor system with improved sensing efficiency through induction of ACEOF to mitigate or obviate the aforementioned problems.

### SUMMARY OF THE INVENTION

The main objective to the present invention is to provide an impedimetric biosensor system, which is economic and laborsaving for use, and has an improved efficiency on detecting a target analyte.

The impedimetric biosensor system comprises a chip and an electrochemical sensor. The chip includes a substrate and at least one electrode assembly. The electrode assembly is mounted on the substrate as working electrodes for contacting an analyte. The electrode assembly is controlled under a controlling condition for ACEOF, such that an alternating current electroosmotic vortex (ACEO vortex) occurs to increase collision between a target in the analyte and the at least one electrode assembly.

According to the present invention, the electrochemical sensor is any equipment or device capable of detecting variance before and after an electrochemical reaction.

Preferably, the electrochemical sensor detects resistance or capacitance of the surface of the working electrodes to acquire electrochemical impedance spectroscopy (EIS).

According to the present invention, each of the at least one electrode assembly has a pair of electrodes, wherein one of the pair of electrodes is a disk electrode and the other of the pair of electrodes is a ring electrode. The disk electrode is round and has a diameter. The ring electrode is in a shape of an arc, surrounds the disk and has a width. A ring-disk distance is formed between the ring electrode and the disk electrode.

Preferably, the ratio of the diameter of the disk electrode to the width of the ring electrode is less than 4:1.

Preferably, the ratio of diameter of the disk electrode to the ring-disk distance is less than 16:1.

Preferably, the ratio of the diameter of the disk electrode to the disk-ring distance to the width of the ring electrode ranges from 400:50:100 to 800:50:100.

Preferably, the controlling condition for ACEOF includes an alternating amplitude ranging from 0.5 Vₚ₋ₚ to 3 Vₚ₋ₚ, and a frequency ranging from 100 Hz to 1 kHz.

Preferably, the analyte is in a solution with a conductivity ranging from 1.24 μS/cm to 840 μS/cm. More preferably, the analyte is in a solution with a conductivity ranging from 1.24 μS/cm to 150 μS/cm.

Preferably, a surface of the electrode assembly is immobilized with a probe, and the probe and the analyte have bioaffinity with each other.

Preferably, the probe is nucleic acid and the analyte is nucleic acid complementary to the probe. More preferably, the probe is polynucleotide and the analyte is polynucleotide complementary to the probe.

In another aspect, the present invention also provides a method for using the impedimetric biosensor system as described above.

The method in accordance with the present invention comprises the steps of providing the impedimetric biosensor system as described above, contacting the at least one electrode assembly with an analyte, wherein the at least one electrode assembly as the working electrodes is controlled under a controlling condition for ACEOF, such that an ACEO vortex occurs to increase collision between a target in the analyte and the at least one electrode assembly; and detecting resistance or capacitance of the surface of the at least one electrode assembly.

According to the present invention, the impedimetric biosensor system is formed by integrating the electrode assembly with the electrochemical sensor to concurrently generate ACEOF and detect resistance or capacitance of the surface of the working electrodes to acquire EIS for determining the presence of a target analyte, which is preferably in conjunction with the particular design of the electrode assembly and the controlling condition for ACEOF with certain operating parameters such as driving voltage and frequency. It is proven that the impedimetric biosensor system in accordance with the present invention has an improved sensing efficiency and lowered detection limitation for detecting biomolecules in an economic and effective way and does not breakdown the biomolecules by routine use, since analyte in a solution with a lower conductivity is allowable to be used in the system or method in accordance with the present invention to generate a mild environment. The impedimetric biosensor system in accordance with the present invention is beneficial to miniature and large-scale production of biosensors with lower detection limitation.

Other objectives, advantages and novel features of the invention will become more apparent from the following detailed description when taken in conjunction with the accompanying drawings.

### BRIEF DESCRIPTION OF THE DRAWINGS

FIG. 1a is Nyquist plot consisting of an imaginary part and a real part of impedance; FIG. 1b illustrates total impedance including real and imaginary parts at a unit of Ω at various scanning frequencies; and FIG. 1c illustrates phase angle of a corresponding impedance component at various scanning frequencies, wherein rectangles represent the experimental group with diffusion, and triangles represent that without diffusion;

FIG. 2a is an illustrative plot of equivalent circuit of the electrode/electrolyte interface with diffusion reaction, and FIG. 2b is an illustrative plot of equivalent circuit of the electrode/electrolyte interface without diffusion reaction, wherein Rₛ represents equivalent resistance of solution, CPE represents equivalent capacitance of EDL, Rₑₜ represents electron transfer resistance and Z_{w} represents impedance caused by diffusion;

FIG. 3 is an illustrative plot of ITO electrode assembly having a disk electrode and a ring electrode with disk diameter (D_{disk}) to be 1000 μm, interelectrode gap (D_{gap}) to be 50 μm, ring width (W_{ring}) to be 500 μm, and M indicates a standard bar representing 500 μm;

FIG. 4 illustrates the relation between average flow rate and frequency at various conductivities (1.2 μS/cm, 6.1 μS/cm and 110 μS/cm) under a condition of driving voltage of 3 Vₚ₋ₚ with the electrode assembly having D_{disk} of 1000 μm, D_{gap} of 50 μm and W_{ring} of 500 μm;

FIG. 5a is an illustrative plot collecting of fluorescence beads on the surface of ring-disk electrodes after performing ACEO stirring for 2 minutes at 3 Vₚₚ and 200 Hz in 1 mM Tris solution, wherein dotted line and dash lines show the edge of disk electrode and ring electrode, respectively. S: the stagnation point as shown with light dot-dashed line;

FIG. 5b is an illustrative side-view scheme of rotating vortices induced by ACEO above the electrodes;

FIGS. 5c to 5d are diagrams illustrating fluorescent intensities of beads collected on the surface of disk electrode of the asymmetric ring-disk electrodes of 1:4, 1:6 and 1:8 W_{ring}-to-D_{disk} ratio, respectively;

FIG. 6 is an illustrative plot of multiple electrode assemblies having gold thin-film electrodes of 4×4 array in a disk-and-ring pattern with D_{disk} of 200 μm, D_{gap} of 50 μm and W_{ring} of 100 μm;

FIG. 7 illustrates effects of applied voltage on the change in electron transfer resistance (ΔRₑₜ) before and after 150 seconds ACEO driving of 200 Hz for bare gold film electrodes and cpDNA/mercaptohexanol (MCH)-modified gold electrodes, each measurement with at least three repetitions;

FIG. 8a illustrates effects with (curves (3), (5) at 200 Hz and curve (4) at 400 Hz) and without (curves (1)-(2)) ACEO stirring of 1.5 Vₚₚ on the hybridization time estimated by the Rₑₜ change (ΔR_{et-dsDNA}) before and after dsDNA formation, wherein the hybridization solution of Curve 1 and Curves 2 to 5 was 10 mM Tris-HCl (pH 7.0) buffer containing 1 M NaCl (designated as Tris (NaCl)) and 1 mM Tris (pH 9.3), respectively, Curves 1 to 4 and Curve 5 show the hybridization of cpDNA/MCH-modified electrodes to 1 nM detected target-DNA (dtDNA) and 1 nM mismatched-dtDNA (mtDNA), respectively;

FIG. 8b illustrates the ΔR_{et-dsDNA} value as a function of dtDNA concentration with 120 seconds ACEO stirring of 200 Hz and 1.5 Vₚₚ for each hybridization concentration; and

FIG. 9 is an illustrative plot of the principle of ACEOF, wherein the dot line refers to electric field, positive mark refers to cation, negative mark refers to anion, hollow arrow refers to direction of ACEO vortex and dash line refers to Coulomb force.

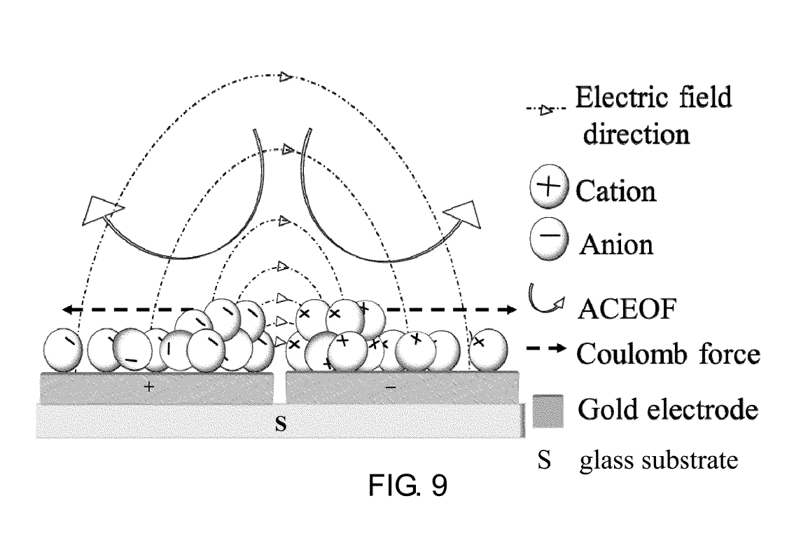

### DETAILED DESCRIPTION OF THE PREFERRED EMBODIMENTS

General Material and Experimental Equipments

1. Reagents

Sodium phosphate dibasic [Na₂HPO₄, M.W. 156.01], sodium phosphate monobasic dihydrate [NaH₂PO₄.2H₂O, M.W. 141.96], hydroxylmethyl aminomethane (Tris) [NH₂C(CH₂OH)₃, M.W. 121.14], sulfuric acid (H₂SO₄, M.W. 98.08), nitric acid (HNO₃, M.W. 63.01), mercaptohexanol (MCH) (HS(CH₂)₆OH, M.W. 134.24), N-[Tris(hydroxymethyl)methyl]-2-aminoethane sulfonic acid (TES) (C₆H₁₅NO₆S, M.W. 229.25), 2-[2-(Bis(carboxymethyl)amino)ethyl-(carboxymethyl)amino]acetic acid (EDTA) (M.W. 292.24), fluorescence-labeled carboxylate-modified polystyrene and latex beads with mean diameter of 1.0 μm (2.5% solids; 0.9-1.5 g/mL; L1030, yellow-green) were purchased from Sigma. Hydrochloric acid (HCl, M.W. 36.48), sodium chloride (NaCl, M.W. 58.5), potassium hexacyanoferrate (III) [K₃Fe(CN)₆, M.W. 329.24] and potassium hexacyanoferrate (II) trihydrate [K₃Fe(CN)₆.3H₂O], M.W. 422.39] were purchased from Showa. Isopropanol (IPA) [(CH₃)₂CHOH, M.W. 60.1] and acetone (CH₃COCH₃, M.W. 58.08) were purchased from Meledla. All chemicals were of reagent grade.

Phosphate buffered solution (PBS) was prepared by dissolving equal amounts of NaH₂PO₄ and Na₂HPO₄ (Sigma) in distilled deionized water (ddH₂O). pH value of PBS was adjusted with NaOH for obtaining proper electric conductivity suitable for preserving biomaterials and ready for being used as analysis buffer.

Hydroxylmethyl aminomethane (Tris) [NH₂C(CH₂OH)₃, M.W. 121.14], (Sigma) was prepared and adjusted to certain pH value by HCl or glycine suitable for preserving biomaterials and ready for being used as analysis buffer.

Sulfuric acid was used to prepare piranha solution. Hydrochloric acid and sodium hydroxide were used to adjust pH value. Nitric acid and hydrochloric acid was used to prepare aqua regia. Isopropanol and acetone were used to clean electrodes. MCH was used in the following examples for blocking area without bounding of modifier and competing with nonspecifically absorbed single strain DNA. TES was adjusted to pH 7 by NaOH and used as analysis buffer.

Potassium hexacyanoferrate (III) was dissolved in 10 mM TES to form a solution at a concentration of 5 mM. Potassium hexacyanoferrate (II) trihydrate was dissolved in 10 mM TES to form a solution at a concentration of 5 mM.

Fluorescence-labeled carboxylate-modified polystyrene and latex beads were characterized in having the following particulate physical properties: permittivity to be 2.53˜2.55 at 25° C., index of refraction to be 1.59 at 590 nm and an initial concentration of 10⁹/ml; and stored at 4° C. before being dispensed into solutions with different electric conductivities and being used in ACEO driving and quantification.

1.2 Experimental Equipments

(1) Spin Coater

Spin coater is commercially available from King Polytechnic Engineering Co., Ltd. (Taiwan) and used to spin-coat the photoresist on the substrate at any spinning rate, time and acceleration.

(2) Mask Aligner:

The mask aligner is commercially available from M & R Nano Technology Co., Ltd. (Taiwan) by the catalog no. AG350-4N-S-S-S-H. The mask aligner is allowed to be adjusted in exposure time and dose, and useful for aligning the mask and the chip and checking microelectrode obtained by photolithography.

(3) Microbalance

The microbalance is commercially available from Metter Toledo by catalog no. AL104 for weighting chemicals used in the following example.

(4) pH Meter:

The pH meter is commercially available from Jenco by catalog no. 6173pH+R for measuring the pH value of solutions and samples.

(5) Impedance Analyzer:

The impedance analyzer is commercially available from IM-6 impedance analyzer having detecting modules for EIS analysis and cyclic voltammery (CV). The impedance analyzer is used for evaluating the surface state of the electrodes and equipped with Thales analogue electronics to simulate corresponding parameter of any circuit component.

(6) Temperature-Controlled Water Incubator:

The temperature-controlled water incubator is commercially available from BRASON and used for heating solutions or DNA for electroplating or hybridization.

(7) Fluorescence Microscope:

The fluorescence microscope is commercially available from Olympus by catalog no. IX71 with an upright and an inverted halogen light sources and a fluorescence mercury light source for observation of different samples and stirring of suspended beads or DNA timely.

(8) Camera:

The camera is commercially available from Olympus by catalog name DP71 for photographing microelectrodes and fluorescence-labeled beads.

(9) Function Generator:

The function generator is Agilent 33220A function generator, which provides 11 standard waves as well as pulse and additional arbitrary waves, providing stable frequency and low distortion in the electrical controlling.

(10) High-Speed Centrifugation:

The high-speed centrifugation is commercially available from Hettich by catalog no. EBA21 for collecting DNA.

(11) Temperature-Controlled Water Incubator:

The temperature-controlled water incubator is commercially available from Major Science (Taiwan) and used for heating cooled DNA.

(12) Laminar Flow:

The laminar Flow is commercially available from Tsao Hsin Enterprise Co., Ltd. (Taiwan) and used for dispensing DNA into multiple aliquots and adjusting to an appropriate concentration.

(13) Conductivity Meter:

The conductivity meter is commercially available from Eutech by catalog no. con510 for measuring conductivity of the solutions used in electric controlling and hybridization.

(14) ITO Transparent Conductive Glass Substrate:

The ITO transparent conductive glass substrate is commercially available from Anatech Co (Taiwan) by catalog no. code007 with a thickness of 7 mm and toping with a polished thin film of ITO with a thickness of 260±20 nm and a sheet resistance less than 7 Ωcm⁻¹. It is used for quantifying the flow rate of fluorescence beads within the disk and ring electrodes.

2. Procedure for Detecting Nucleic Acid

In the example, the nucleic acids were commercially available from Bio Basic Inc. with HPLC purification. 105 μL of D.I. water was dropped into the tube containing DNA powder. DNA attached to the wall of the tube was detached therefrom by centrifugation to form a DNA solution at a concentration of 100 μM, further being adjusted to 1 μM with Tris (NaCl).

Gold electrodes were cleaned in turn with Piranha solution (7:3, v/v, H₂SO₄ (conc.): H₂O₂ (30%)) for 2 minutes and aqua regia (3:1, HCl: HNO₃) for 1 minute, followed by electrochemical cleaning of cyclically sweeping from 0 to −1.5V in 10 mM PBS till a consistent wave shape was obtained and ready to be used as electrodes for the following detection of DNA.

Procedure:

Stage 1: 15 μl aliquot of 1 μM cpDNA solution prepared in 10 mM Tris-HCl (pH 7.0) buffer containing 1 M NaCl (designated as Tris-HCl (1 M NaCl)) was placed on the cleaned gold electrodes for 2 hours to form a self-assembled monolayer. The gold electrodes were rinsed with 20 μl Tris (NaCl) and D.I. water for multiple times.

Stage 2: After rinsing out the unbound cpDNA with pure water, 20 μl 1 mM MCH prepared in pure water was dropped on the cpDNA-immobilized electrodes for 1 hour to prevent the electrode surface from the non-specific adsorption of cpDNA to form cpDNA/MCH-modified electrodes.

Stage 3: The cpDNA/MCH-modified electrodes can be hybridized with the 20 μl concentration-varied detected target-DNA (dtDNA) prepared in Tris (NaCl) or 1 mM Tris (pH 9.3) without the application of ACEO stirring, followed by being subjected to emersion in 250 μL of 10 mM Tris-HCl (pH 7.0) for 10 minutes to remove unbound dtDNA therefrom and ready for EIS measurements and CV quantification.

Stage 3′:20 μl of 1 μM dtDNA or mismatched-dtDNA (mtDNA) prepared in 1 mM Tris was dropped on the cpDNA/MCH-modified electrodes under application of AC stirring for 120 seconds.

The sequences of the nucleic acids are listed in the following Table 1.

TABLE 1
Probe and target gene sequences

| | Sequence |
|---|---|
| complementary | 5′-SH-(CH₂)₆-CAC ACC TGA CTT GAC AGA CC-3′ |
| probe-DNA | (SEQ ID NO. 1) |
| (cpDNA) | 20 base single strain DNA derived from Salmonella |
| | typhmurium Stml 16S rRNA gene and modified with |
| | SH-(CH₂)₆ group at 5′ end |
| detected | 5′-GGT CTG TCA AGT CAG GTG TG-3′ (SEQ ID NO. 2) |
| target-DNA | 20 base single strain DNA derived from Salmonella |
| (dtDNA) | typhmurium and perfectly complementary to cpDNA |
| mismatch-dtDNA | 5′-GGT CTG TCA A [symbol] T CAG GTG TG-3′ (SEQ ID NO. 3) |
| (mtDNA) | The sequence of single base mismatch tDNA (designated |
| | as mtDNA) at position 11 (T instead of G) counted from |
| | 5′-end |

3. CV and EIS Measurements

3.1 Formulation of Analysis Buffer

In the experimental procedure of the following example, 5 mM

Fe(CN)₆^{3-/4-} in 10 mM TES buffer at pH 7 with an electric conductivity of 4.08 mS/cm was used for hybridization with or without ACEO vortex and subjected to CV and EIS measurements.

3.2 Condition and Analysis by CV and EIS Measurements

All electrochemical measurements were carried out with the IM-6 impedance analyzer (Zahner Electrik GmbH, Germany) CV and EIS were fulfilled in a conventional three-electrode cell. The disk gold electrode array, an Ag/AgCl (MF2052, Bioanalytical Systems Inc., West Lafayette, Ind.) and a Pt wire were used as working electrode, reference electrode and counter electrode, respectively. An equimolar Fe(CN)₆^{3-/4-} mixture (5 mM) in 10 mM TES buffer (pH 7.0) was used to explore the electrochemical properties of electrode/electrolyte interface. A cyclic voltage ranging from −0.1 V to +0.5 V at the scan rate of 20 mV/s was used to measure the redox current of Fe(CN)₆^{3-/4-} mediators. Impedimetric measurement was carried out in a frequency ranging from 1 Hz to 100 kHz at a +0.21 V voltage added with a 5 mV amplitude sine wave. The acquisition and analysis of impedance spectra, and the simulation of equivalent circuits were carried out with the IM-6/THALES software package.

3.3 Design of EIS Equivalent Circuit

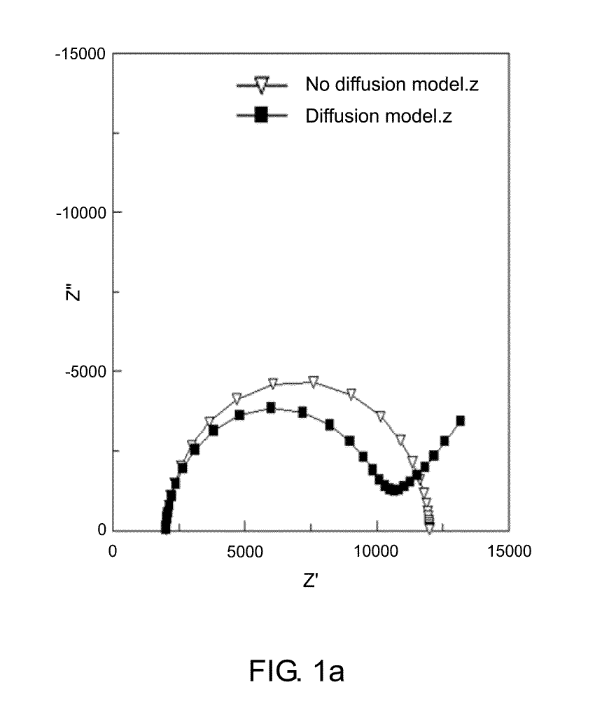

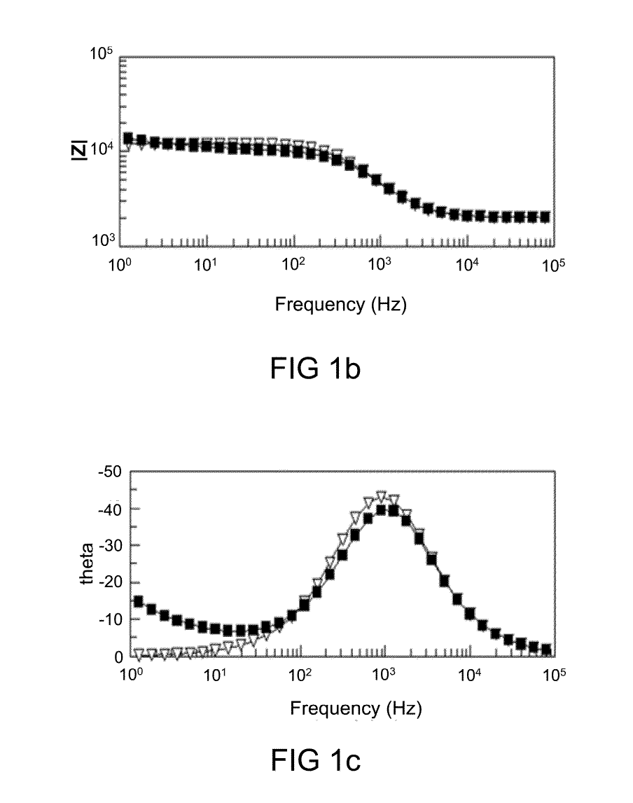

After EIS measurement, Nyquist plots and Bode diagrams corresponding to spectra were obtained (FIG. 1a to 1c). Equivalent circuit was established for simulating the obtained EIS spectra. While the surface modified layer was loose and under a lower frequency, a net oxidation-and-reduction of an electric-active substance occurs, leading to the occurrence of analyte diffusion, wherein the equivalent circuit required Z_{w} component for simulating diffusion impedance as shown in FIG. 2a. On the other hand, while there existed modification with modifiers such as DNA or MCH, no diffusion occurred as marked as triangle in Nyquist plot, Z_{w} was deleted and Rₑₜ was connected to CPE in parallel for simulation as shown in FIG. 2b.

### Example

First, velocity of the ACEOF and the preferred range of the working frequency in solutions with various electric conductivities were explored in the present example. All electrode assemblies have a constant interelectrode gap (D_{gap}) being 50 μm. For precisely quantifying the velocity of ACEOF, transparent indium tin oxide (ITO) electrodes and optical methodology were utilized for exploring the velocity of ACEOF under various conductivities.

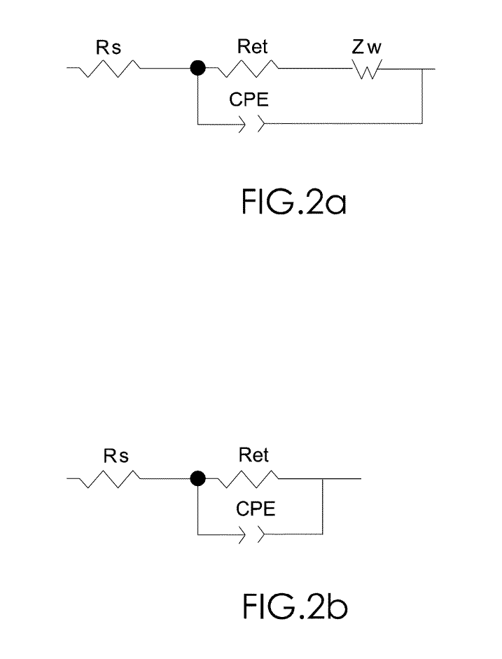

ITO electrodes in a ring-and-disk pattern were manufactured by microelectromechanical processes, including defining sacrificial layer of positive photoresist (S1818, Microchem) by photolithography, depleting unprotected ITO area, depleting sacrificial layer of positive photoresist, and finally applying negative photoresist (SU8-3010, Microchem) as insulating layer 30 to define a working electrode area and forming a disk electrode 10 and a ring electrode 20 as shown in FIG. 3.

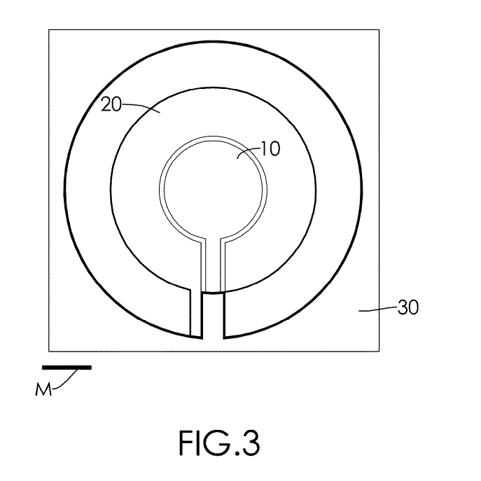

The obtained electrodes were used for exploring optimal velocity of ACEOF in solutions with conductivities of 1.2 μS/cm, 6.1 μS/cm and 110 μS/cm at 3Vₚ₋ₚ. The velocity of ACEOF was determined as the moving speed of fluorescence-labeled polystyrene beads during 0.5 to 1.0 second after its passing through the edge of disk electrode. As shown in FIG. 4, the results demonstrated that in solutions with conductivities of 1.2 μS/cm, 6.1 μS/cm and 110 μS/cm the optimal ranges of the frequency of ACEO driving were 75, 150 to 200, and 600 to 700 Hz respectively.

Subsequently, for quantification of collection of fluorescence beads, 10⁶ particles/mL fluorescence bead suspension as described in “general material and experimental equipment” was applied onto ITO electrode assemblies having the disk and ring electrodes with the same D_{gap} of 50 μm, the same W_{ring} of 100 μm, and a different D_{disk} of 400 μm, 600 μm or 800 μm. The fluorescence intensity on the disk electrode controlled by ACEOF under a condition of 6.1 μS/cm, 3Vₚ₋ₚ and 200 Hz was determined by counting numbers of fluoresces beads settling down on the disk electrode after ACEOF driving for 2 minutes by using the fluorescence microscope as described in “General material and experimental equipment” and analyzed by ImagJ software (Research Services Branch, National Institute of Mental Health, Bethesda, Md., U.S.A).

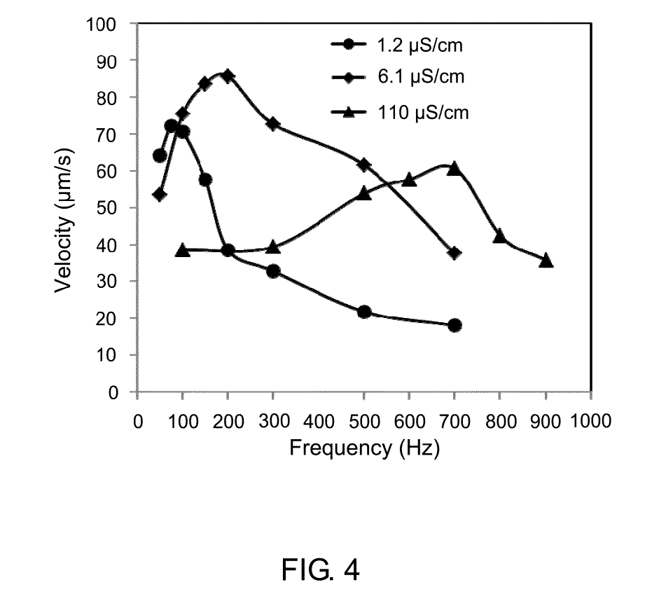

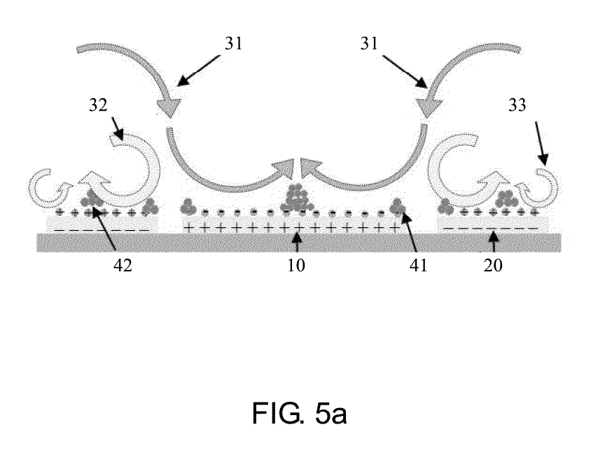

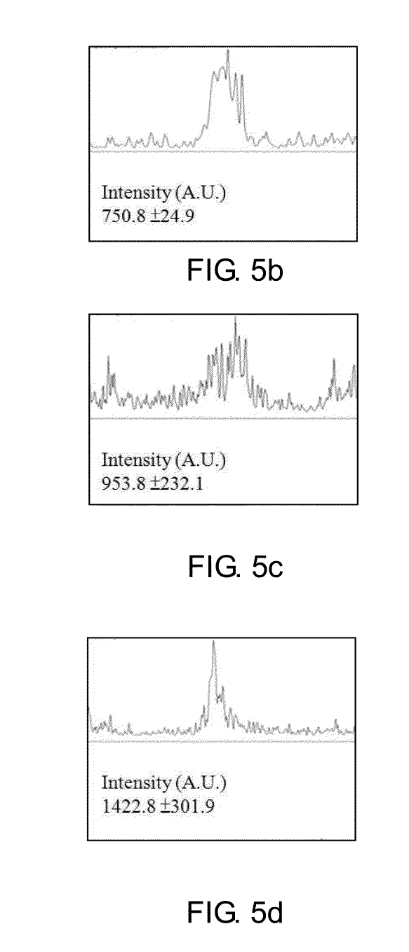

The illustrative diagram of fluorescence beads settling down on the electrode assemblies after ACEOF stirring for 2 minutes and ACEOF occurring on the disk and ring electrodes was shown in FIG. 5a. In a geometric asymmetric electrode set, there is a unidirectional flow above the disk electrodes (10) to move fluorescence beads from small ring electrode (20) to large disk electrodes (10). Generally, the fluid above the disk electrode (10) experiences the large tangential electric field to form a slow and large ACEO fluid roll (31). The fluorescence beads were attracted to the edge of disk electrode (10) and ring electrode (20) due to the attraction of positive DEP (41), and most fluorescence beads can be collected in the center of disk electrode (10) due to ACEO driving. Besides, the fluorescence beads on the ring electrode (20) were also collected at a fixed position, called stagnation point (42), resulting from the changes in tangential electric fields. Generally, the fluid near the inner edge of ring electrode (20) experiences the larger tangential electric field to form a fast and small ACEO fluid roll (32), and the fluid near the outer edge of ring electrode (20) experiences the smaller tangential electric field to form a slow and small fluid roll (33). When the two counter rotating vortices meet at the stagnation point (42), the fluorescence beads were precipitated and aggregated on the surface of ring electrode (20).

As shown in FIGS. 5b to 5d, the mean fluorescent intensity (arbitrary unit, A.U.) of beads collected on the surface of disk electrode with the D_{disk} to W_{ring} ratio of 4:1, 6:1 and 8:1 was 750.8, 953.8 and 1422.8, respectively, after ACEO driving for 2 minutes. The ratio of fluorescence intensities normalized by the area of disk electrode on the electrode assemblies with the D_{disk} to W_{ring} ratio of 4:1, 6:1 and 8:1 was 2.11:1.19:1.00, respectively. This result indicated that the smaller the ratio of the diameter of disk electrode (D_{disk}) to the width of ring electrode (W_{ring}) was, the more beads aggregated per the same unit area of the disk working electrode. Most of the beads were collected at the center of the disk electrode by ACEOF, demonstrating that the analyte distant from the surface of the electrode can be led by the ACEOF vortex to gather at the center of the disk electrode. Thus, the probability of collision between analyte and suggestive biosensing layer on the modified surface of the electrode could be promoted. The results also suggested that the electrode assembly of the disk and ring electrodes with a smaller ratio of D_{disk} to W_{ring} had better collecting efficiency.

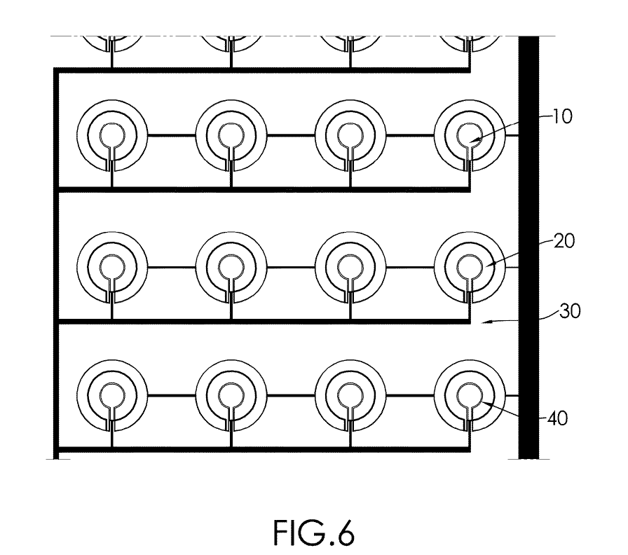

In an embodiment, a chip having gold film electrodes (gold electrodes), which are electrode assemblies with a disk electrode 10, an interelectrode gap 40, and a ring electrode 20 made of gold with D_{disk} being 400 μm, D_{gap} being 50 μm and W_{ring} being 100 μm as shown in FIG. 6 were prepared by the steps as follows.

(1) Glass substrate was immersed into D.D. water and sonicated for 5 minutes for three times. The washed glass substrate was then dried and sonicated in IPA for 5 minutes repeatedly for 3 times, followed by removal of IPA and drying. The dried glass substrate was placed into piranha solution and heated to 80° C. and sonicated for 5 minutes for 3 to 5 times, followed by removal of piranha solution to obtain a cleaned glass substrate. The cleaned glass substrate was dried by heating at 95° C. for 5 minutes.

(2) The glass substrate was spin-coated with a positive photoresist (AZ44620, Shipley) by 500 rpm for 10 seconds at the first spin and 3000 rpm for 40 seconds at the second spin to obtain a 2 μm-thick positive photoresist layer. The spin-coated glass substrate was subjected to a soft bake at 95° C. for 10 minutes and annealed to room temperature. The purpose of soft bake was for evaporating the solvent in the photoresist to increase the adhesion between the photoresist and the substrate.

(3) A sacrificial layer of positive photoresist with a corresponding electrode pattern was formed by photolithography, wherein the spin-coated substrate was aligned with a photomask and exposed to UV light at a wave length of 365 nm with an exposure dose of 135 mJ/cm².

(4) Developing reagent was diluted with D.D water at 1:2 and used to develop the exposed photoresist for 2 minutes and then the developed exposed photoresist was washed with D.D water to remove the residual of the developing reagent.

(5) A 20 nm-thick Ti layer as adhesion layer and a 200 nm-thick Au layer were deposited by evaporation or sputtering. The deposited electrodes were immersed in acetone solution to remove the scarified layer to obtain gold film electrodes. For defining the area of the electrode, a negative photoresist was formed on the electrode as insulating layer 30 as shown in FIG. 6. The electrodes were cleaned and baked at 95° C. for 5 minutes for drying. Subsequently, the area of the gold film electrode was measured.

(6) Negative photoresist SU8-3010 was applied to the substrate with the gold film electrodes by spin-coating under a condition of 500 rpm for 10 seconds at the first spin and 2500 rpm for 40 seconds at the second spin to obtain a 6-to-8-μm-thick layer of negative photoresist.

(7) The glass substrate with the layer of the negative photoresist was exposed to UV light at 365 nm for an exposure dose of 320 mJ/cm² to form an exposed chip. The exposed chip was subjected to a post bake on a heater to trigger cross-linking to occur and promote the degree of cross-linkage in the photoresist.

(8) The exposed chip was then developed in the developing reagent for 2 minutes, followed by removal of developing reagent, washing with IPA and hard bake on a heater at 150° C. for 10 minutes in order to enhance the adhesion between the photoresist and the chip. An insulating layer 30 was manufactured. The working area of electrodes including 16 sets of disk and ring electrodes 10, 20 was then defined, wherein the total area of the disk electrodes 10 was 2 mm²; the width of the ring electrode 20 was 100 and the gap 40 of the disk and ring electrodes 10, 20 was 50 μm. Finally, multiple electrode assemblies with gold film electrodes in a disk-and-ring pattern were established as shown in FIG. 6.

The stability of the chip before hybridization was determined as follows. The driving voltage of ACEOF could destroy the electrodes or cause departure of probe from the electrodes by breaking the gold and sulfide bond and absorption by Van der waal force. Therefore, in order to obtain the optimal operational voltage of ACEO, the stabilities of bare gold film electrodes and gold film electrodes as obtained after Stage 1 and Stage 2 of the method as described in “3. Procedure for detecting nucleic acid” were evaluated by various voltages (1 Vₚ₋ₚ, 1.5 Vₚ₋ₚ, 2 Vₚ₋ₚ, 2.5 Vₚ₋ₚ, 3 Vₚ₋ₚ,) under a condition of a frequency of 200 Hz and conductivity of 6.1 μS/cm for 150 seconds.

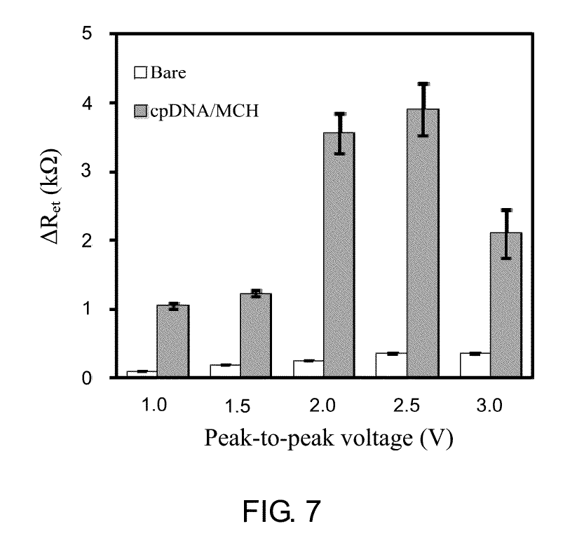

FIG. 7 demonstrated that the changes of Rₑₜ of the disk electrode of the bare gold film electrodes and cpDNA/MCH-modified gold film electrodes before and after application of ACEOF diving. The change of Rₑₜ (ΔRₑₜ) was determined by R_{et-ACEOF}−Rₑₜ₋ᵢₙᵢₜᵢₐₗ, wherein R_{et-ACEOF} represented Rₑₜ of the surface of the electrode after ACEO driving, Rₑₜ₋ᵢₙᵢₜᵢₐₗ represented Rₑₜ of the surface of the electrode before ACEO driving. Results illustrated that ΔRₑₜ was increased with voltage of the ACEO driving. However, the increment of ΔRₑₜ of bare gold film electrode was less than that of the cpDNA/MCH-modified gold film electrodes. With regards to bare gold film electrodes, a gold oxide film was formed after application of ACEO driving. It was observed that bubbles occurred when the applied voltage for ACEO driving was over 3 Vₚ₋ₚ. With regards to cpDNA/MCH modified gold film electrode, applied voltage for ACEO driving might trigger collapse of the complex structure of cpDNA/MCH to result in nonspecific adsorption of cpDNA on the surface of the electrodes, the negative charged phosphate group of the collapsed cpDNA would reduce the permeability of Fe(CN)₆^{3-/4-} on the surface of the electrodes, resulting in increase of Rₑₜ. Especially, when the voltage was increased to 2 Vₚ₋ₚ and 2.5 Vₚ₋ₚ, ΔRₑₜ greatly increased, implying that ACEOF and the applied voltage caused more prominently nonspecific adsorption from collapsed cpDNA/MCH complex structure. When the voltage was increased to 3 Vₚ₋ₚ, ΔR_{et-ACEOF} decreased and water electrolysis occurred. The phenomenon might be attributed to the MCH and cpDNA desorptions, caused by the reductive reaction of Au—S bond breakdown.

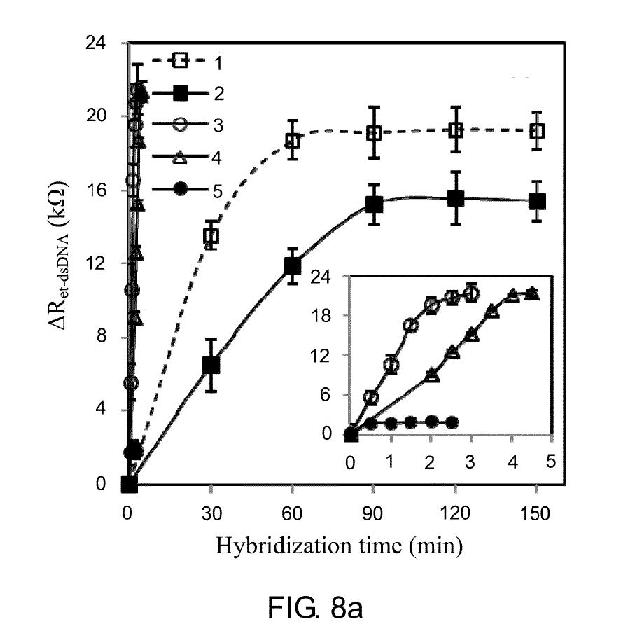

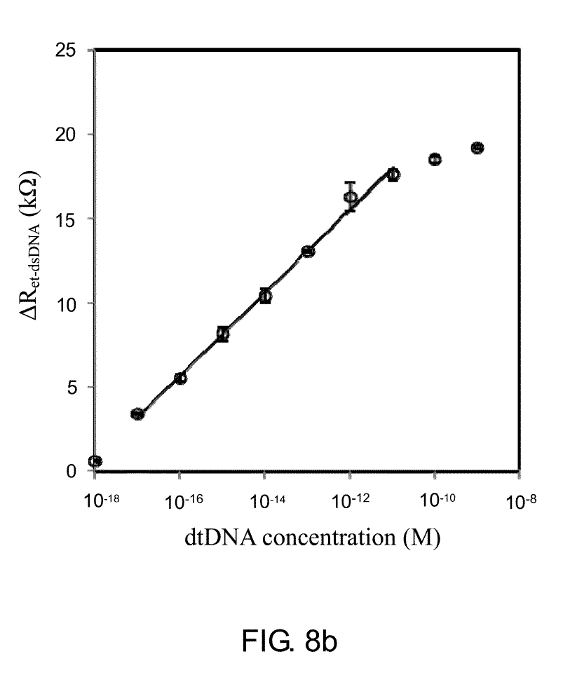

Given that the results were as described above, to evaluate hybridization between detected target DNA (dtDNA) or mismatch detected target DNA (mtDNA) and complementary probe DNA (cpDNA), subsequently used was a chip having 16 electrode assemblies of gold film electrodes in a disk-and-ring electrode pattern with immobilized DNA probe (cpDNA) on the surface of the gold film electrodes through covalent bonding of thiol group to gold surface, wherein the D_{ring} was 400 μm, the W_{ring} was 100 μm and the D_{gap} was 50 μm. In the following embodiment, under a condition of conductivity to be 6.1 μS/cm, applied voltage for ACEOF driving to be 1.5 Vp-p and frequency to be 200 Hz, the hybridization with ACEOF driving was monitored. The hybridization was performed by the procedure Stage 3 or 3′ as described in “3. Procedure for detecting nucleic acid”. The results were shown in FIGS. 8a and 8b.

Curve 1 and Curve 2 in FIG. 8a respectively demonstrated the changes of Rₑₜ of the experimental group of dtDNA in a solution with high conductivity (96 mS/cm) and low conductivity (6.1 μS/cm) without ACEOF. The change of Rₑₜ of dtDNA in a solution, ΔR_{et-dsDNA}, was determined by R_{et-dsDNA}−R_{et-cpDNA/MCH}, wherein R_{et-dsDNA} represented Rₑₜ after hybridization, and R_{et-cpDNA/MCH} represented Rₑₜ before hybridization. The results demonstrated that whether in a solution with high conductivity or low conductivity, hybridization would occur merely by diffusion. In the solution with conductivity of 96 mS/cm, the time for reaching hybridization plateau was 60 minutes. In the solution with conductivity of 6.1 μS/cm, the time for reaching hybridization plateau was 90 minutes. ΔR_{et-dsDNA} in the solution with conductivity of 6.1 μS/cm was less than that of 96 μS/cm, indicating that under a condition of conductivity to be 6.1 μS/cm, less extent of hybridization occurred. Curve 3 and Curve 4 in FIG. 8a respectively demonstrated the changes of Rₑₜ of the experimental group of dtDNA in a solution with low conductivity (6.1 μS/cm) before and after hybridization under a condition of ACEOF at a voltage of 1.5 Vₚ₋ₚ, a frequency of 200 Hz and 400 Hz. 90% response time of saturating hybridization at 200 Hz and 400 Hz were respectively about 117 seconds and 216 seconds, indicating that ACEOF could promote the hybridization rate. Hybridization plateau was reached quicker under the condition of 200 Hz at the conductivity of 6.1 μS/cm than that of 400 Hz. The 90% response time of saturating hybridization at 200 Hz was 0.022 times of that under a condition without ACEOF. Furthermore, both ΔR_{et-dsDNA} plateau values of 200 Hz and 400 Hz were 21.4 kΩ, which was 1.41 times larger than that of hybridization without ACEOF. The results demonstrated that the ACEOF effectively improved the hybridization efficiency.

Curve 5 in FIG. 8a illustrated the kinetic hybridization reaction of mtDNA. The plateau value of ΔR_{et-dsDNA} was 1.8±0.4 kΩ after performing the 200 Hz-driven ACEO for 150 seconds, which is much smaller than the ΔR_{et-dsDNA} plateau value of dtDNA hybridization. Moreover, compared with previous literatures of performing unstirred hybridization for 20 base tDNA of single base mismatch, the ratio of mtDNA-to-dtDNA ΔR_{et-dsDNA} plateau value is 8.4%. The results indicated that hybridization between detected target DNA and its perfectly complementary DNA probe was distinguishable from that between the DNA probe and its single mismatch detected DNA.

FIG. 8b illustrated the calibration curve of concentration-varied dtDNA with 200 Hz-driven ACEO for 120 seconds. To further define the linearity over the 10 aM to 10 pM concentration, the linear regression analysis of ΔR_{et-dsDNA} values against the dtDNA concentration had an equation of ΔR_{et-dsDNA} (kΩ)=2.46 log [dtDNA]+45.04 with R²=0.9953. The high correlation coefficient implied a good linear relationship between the ΔR_{et-dsDNA} values and the dtDNA concentration. The ΔR_{et-dsDNA} value after incubation with 10 aM dtDNA was 3.4±0.2 kΩ. The accuracy of the Rₑₜ (the standard deviation of repeated measurements) of cpDNA/MCH-modified electrodes before dtDNA hybridization was 1.01 kΩ. Therefore, the limit of detection (LOD) was 10 aM. The extreme low LOD is attributed to the ACEO stirring to facilitate the hybridization reaction.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An impedimetric biosensor system, comprising:
a chip, including:
a substrate, and
at least one electrode assembly mounted on the substrate as working electrodes for contacting an analyte, wherein the at least one electrode assembly is controlled under a controlling condition for alternating current electroosmotic flow (ACEOF), such that an ACEO vortex occurs to increase collision between a target in the analyte and the at least one electrode assembly; and
an electrochemical sensor connected to the at least one electrode assembly of the chip to detect resistance or capacitance of the surface of the working electrodes.

2. The impedimetric biosensor system of claim 1, wherein the electrochemical sensor detects resistance or capacitance of the surface of the working electrodes to acquire electrochemical impedance spectroscopy (EIS).

3. The impedimetric biosensor system of claim 1, wherein each of the at least one electrode assembly has
a disk electrode in a shape of a round disk and having a diameter, and
a ring electrode in a shape of an arc, surrounding the disk and having a width, wherein a ring-disk distance is formed between the ring electrode and the disk electrode.

4. The impedimetric biosensor system of claim 3, wherein the ratio of the diameter of the disk electrode to the ring-disk distance is less than 16:1.

5. The impedimetric biosensor system of claim 4, wherein the ratio of the diameter of the disk electrode to the disk-ring distance to the width of the ring electrode ranges from 400:50:100 to 800:50:100.

6. The impedimetric biosensor system of claim 1, wherein the controlling condition for alternating current electroosmotic flow (ACEOF) includes an alternating amplitude ranging from 0.5 Vₚ₋ₚ to 3 Vₚ₋ₚ and a frequency ranging from 100 Hz to 1 kHz.

7. The impedimetric biosensor system of claim 1, wherein the analyte is in a solution with a conductivity ranging from 1.24 μS/cm to 150 μS/cm.

8. The impedimetric biosensor system of claim 1, wherein the surface of the electrode assembly is immobilized with a probe, and the probe and the analyte have bioaffinity with each other.

9. The impedimetric biosensor system of claim 8, wherein the probe is nucleic acid and the analyte is nucleic acid.

10. A method for using the impedimetric biosensor system of claim 1, comprising:
providing the impedimetric biosensor system of claim 1,
contacting the at least one electrode assembly with an analyte, wherein the at least one electrode assembly as the working electrodes is controlled under a controlling condition for ACEOF, such that an ACEO vortex occurs to increase collision between a target in the analyte and the at least one electrode assembly; and
detecting resistance or capacitance of the surface of the working electrodes.

11. The method of claim 10, wherein the detecting resistance or capacitance of the surface of the working electrodes includes detecting resistance or capacitance of the surface of the working electrodes by the electrochemical sensor to acquire electrochemical impedance spectroscopy (EIS).

12. The method of claim 10, wherein each of the at least one electrode assembly has
a disk electrode in a shape of a round disk and having a diameter, and
a ring electrode in a shape of an arc, surrounding the disk and having a width, wherein a ring-disk distance is formed between the ring electrode and the disk electrode, and the disk electrode and the ring electrode are used as working electrodes.

13. The method of claim 12, wherein the ratio of the diameter of the disk electrode to the ring-disk distance is less than 16:1.

14. The method of claim 13, wherein the ratio of the diameter of the disk electrode to the disk-ring distance to the width of the ring electrode ranges from 400:50:100 to 800:50:100.

15. The method of claim 10, wherein the controlling condition for alternating current electroosmotic flow (ACEOF) includes an alternating amplitude ranging from 0.5 Vₚ₋ₚ to 3 Vₚ₋ₚ and a frequency ranging from 100 Hz to 1 kHz.

16. The method of claim 10, wherein the analyte is in a solution with a conductivity ranging from 1.24 μS/cm to 150 μS/cm.

17. The method of claim 10, wherein the surface of the electrode assembly is immobilized with a probe, and the probe and the analyte have bioaffinity with each other.

18. The impedimetric biosensor system of claim 17, wherein the probe is nucleic acid and the analyte is nucleic acid.


SEQUENCE LISTING (3 sequences)

SEQ ID NO: 1 - DNA, 20 bp, artificial
  20 base single strain DNA derived from Salmonella typhmurium Stml 16S rRNA gene and modified with SH-(CH2)6 group at 5′end
cacacctgacttgacagacc

SEQ ID NO: 2 - DNA, 20 bp, artificial
  20 base single strain DNA derived from Salmonella typhmurium and perfectly complementary to cpDNA
ggtctgtcaagtcaggtgtg

SEQ ID NO: 3 - DNA, 20 bp, artificial
  The sequence of single base mismatch tDNA (designated as mtDNA) at position 11 (T instead of G) counted from 5′-end
ggtctgtcaattcaggtgtg